(19) United States
(12) Patent Application Publication
(10) Pub. No.: US 2010/0145471 A1
(43) Pub. Date: Jun. 10, 2010

(54) PROSTHETIC LUNG

(75) Inventor: William Richard Johns, Reading (GB)

Correspondence Address: EDWIN D. SCHINDLER, FIVE HIRSCH AVENUE, P.O. BOX 966, CORAM, NY, 11727-0966, US

(73) Assignee: University of Vermont and State Agricultural College, Burlington, VT (US)

(21) Appl. No.: 12/530,288

(22) PCT Filed: Mar. 7, 2008
(86) PCT No.: PCT/GB2008/050164

§ 371 Date: Jan. 5, 2010

(30) Foreign Application Priority Data
Mar. 8, 2007 (GB) 0704479.5

(51) Int. Cl. A61F 2/04; A61M 1/16

(52) U.S. Cl. 623/23.65

(57) ABSTRACT

A prosthetic lung for receipt by a lung space of a patient includes a mass exchange apparatus for use in blood/air mass exchange, an air sac and an air vessel. The mass exchange includes plural blood flow conduits for defining blood flow and a plural air flow conduits for defining air flow. The plural air flow conduits and the plural blood flow conduits at least partially include gas-permeable membrane material and the conduits are arranged relative to each other to enable transfer of oxygen from the air to the blood and transfer of carbon dioxide from the blood to the air. The mass exchange apparatus is provided with at least one first air port and at least one second air port, so that the air flow may be defined therebetween by the plural air flow conduits. The air sac defines an air sac cavity in fluid communication with at least one first air port of the mass exchange apparatus. The air vessel defines an air vessel cavity in fluid communication with at least one second air port of the mass exchange apparatus. The air vessel is provided with an air access port arranged, in use, to enable air flow communication with the trachea of the patient.

### TECHNICAL FIELD

The present invention relates to a prosthetic lung including a blood/air mass exchange apparatus and suitable for use internally within the body of a patient.

### BACKGROUND TO THE INVENTION

In Europe and North America, there are currently about 10,000 people on lung-transplant waiting lists. Each year, about 2500 people are transplanted, of whom approximately 2000 survive to live healthy lives. Each year about 2500 die on the waiting list, during a typical 2-year waiting period. The situation is actually far worse than the statistics would indicate because a much larger number of people are never entered onto waiting lists. These people may be excluded because they have no chance of surviving the wait for a transplant or because they are too old. There is little prospect that the situation will improve because the availability of donor organs is declining.

The controversial solution of xeno-transplantation appears to remain in the distant future. The availability of suitable prosthetic lungs would revolutionize the situation. The clinical trials requirements are likely to be more straightforward for prosthetics than for xeno-transplantation, and consequently, the potential time scale for introduction of prosthetic lungs is likely to be shorter. To date, the development of prosthetic lungs has been deterred because of the perceived difficulty involved in reproducing the structure and function of a human lung.

It is known that human lungs have a complex system of branching tubes leading to a multiplicity of small air sacs in which counter-diffusion (oxygen with carbon dioxide) takes place. The Applicant has realized that the engineering challenge in reproducing this kind of structure precludes any prosthesis that directly mimics the human lung.

Applicant's earlier published PCT Patent Application No. W02005/118025 describes a prosthetic lung having a structure that is simpler than that of a human lung, but capable of comparable respiratory function. This prosthetic lung comprises a mass exchange apparatus that functions as a counter-diffusion device to transfer oxygen from the air into the blood and carbon dioxide from the blood to the air. The blood and air flow in alternate channels or conduits. The walls defining the channels or conduits are gas-permeable membranes, which allow oxygen and carbon dioxide to diffuse in opposite directions. The blood flows in one direction through the mass exchange apparatus. Air may flow in alternate directions (as in normal breathing) or in directions controlled by fluidic components. This prosthetic lung also comprises an air sac for supplying air flow to the air flow conduits.

Applicant has now devised a variation and improvement to the prosthetic lung described above, which provides for better control of blood gas concentrations, and hence potentially provides enhanced patient treatment. The improvement involves the provision of an air sac and an air vessel such as to define an air sac cavity and an air vessel cavity. The air sac cavity is arranged for fluid communication with at least one first air port of the mass exchange apparatus and the air vessel cavity is arranged for fluid communication with at least one second air port of the mass exchange apparatus. The air vessel is also provided with an air access port arranged in use, to enable air flow communication with the trachea of the patient, and hence with the outside atmosphere via the trachea, nose and mouth. Thus, all or a proportion of any air that moves from the air vessel cavity to the air sac cavity has to pass through the mass exchange apparatus.

It is an object of the present invention to provide an improved prosthetic lung for use in a human (or other mammalian) body.

### SUMMARY OF THE INVENTION

According to a first aspect of the present invention there is provided a prosthetic lung for receipt by a lung space of a patient comprising

(a) a mass exchange apparatus for use in blood/air mass exchange comprising
(i) plural blood flow conduits for defining blood flow; and
(ii) plural air flow conduits for defining air flow;
wherein said plural air flow conduits and said plural blood flow conduits at least partially comprise gas-permeable membrane material, and the conduits are arranged relative to each other such as to enable transfer of oxygen from the air to the blood and transfer of carbon dioxide from the blood to the air through said membrane material,
and wherein the mass exchange apparatus is provided with at least one first air port and at least one second air port such that said air flow may be defined between said at least one first air port to the at least one second air port via the plural air flow conduits;
(b) an air sac defining an air sac cavity in fluid communication with the at least one first air port of the mass exchange apparatus; and
(c) an air vessel defining an air vessel cavity in fluid communication with the at least one second air port of the mass exchange apparatus, said air vessel provided with an air access port arranged in use, to enable air flow communication with the trachea of the patient.

There is provided a prosthetic lung for use within a human (or other mammalian) body. In use, the prosthetic lung is arranged for receipt by a lung space of a patient.

The prosthetic lung herein includes at least one mass exchange apparatus for use in blood/air mass exchange comprising

(i) plural blood flow conduits for defining blood flow;
(ii) plural air flow conduits for defining air flow;

The plural air flow conduits and the plural blood flow conduits at least partially comprise gas-permeable membrane material, and the conduits are arranged relative to each other such as to enable transfer of oxygen from the air to the blood and transfer of carbon dioxide from the blood to the air through said membrane material.

The mass exchange apparatus is provided with at least one first air port and at least one second air port such that an air flow may be defined between said at least one first air port to the at least second air port via the plural air flow conduits.

The term ‘air port’ herein is used to generally mean an opening provided to the mass exchange apparatus and through which air may flow. In use, and as will become clearer from the later description, each ‘air port’ may function as either as air inlet or air outlet depending upon the mode of operation of the mass exchange apparatus.

Within the mass exchange apparatus, the blood and air do not directly come into contact.

It will be appreciated that the walls defining the blood flow and air flow conduits may be separately formed and arranged relative to each other to enable the necessary exchange of air and carbon dioxide.

In one aspect, the blood and air flow conduits share at least some common walls, again with the arrangement selected to enable the necessary exchange of air and carbon dioxide.

Suitably, the blood flow conduits and/or air flow conduits have a diameter (or cross-section of non-circular conduit) of less than 0.5 mm.

The walls defining the blood and air flow conduits suitably comprise gas-permeable membrane materials for the walls defining the blood and air flow conduits. Such gas-permeable membrane materials may comprise conventional materials (e.g. polymers) or composite materials. A composite material may comprise of two components, a first material component of the composite provides physical strength and a second material component provides gas permeability.

Suitable gas-permeable membrane materials for the walls are biocompatible in nature.

By way of background it is noted that the design of the mass exchange apparatus herein is suitably arranged to minimize the possibility of the generation of blood clots, which might risk the life of the patient. The natural behaviour of blood is to clot when it contacts any surface other than it expects to contact naturally within the body. Specifically, it does not normally clot within blood vessels. This clotting behaviour is essential to avoid haemorrhage whenever there is a cut or bruise. Biocompatible materials for use in the mass exchange apparatus herein desirably achieve biocompatibility by presenting a suitable surface to the blood. Not only are the gas-permeable membrane materials herein suitably biocompatible, but also the tubing connecting the patient with the apparatus and any blood pumps and valves.

Preferably, all valves are in contact only with air (or the oxygen and carbon dioxide containing fluid used instead of air).

In aspects, the mass exchange apparatus herein can be made from any materials widely used in medicine. The patient would take anti-coagulant medication to avoid clots forming. However, use of anticoagulants presents a risk of haemorrhage. Hence, it is desirable to employ materials such that, even in the absence of anticoagulants, blood clots do not form in the mass exchange apparatus. The incentive to employ such anti-clotting materials is particularly important in such an apparatus intended for medium to long-term use. Generally, the anti-clotting property is introduced by applying a coating to surfaces that contact blood. In aspects, the gas-permeable membrane materials herein are subjected to suitable surface treatment thereof.

In one aspect, the gas-permeable membrane materials present an inert surface that results in minimal interaction with the blood. Suitable inert materials can be hydrophilic or hydrophobic, can have a surface that tightly binds water, or can have a surface that mimics the endothelial cells coating the inside of natural blood vessels.

In another aspect, the gas-permeable membrane materials incorporate an anti-thrombogenic agent (or agents) in their surface. Materials that incorporate anti-thrombogenic agents most frequently have heparin (or a heparin derivative) bound to the surface. Heparin may suitably be bound covalently or ionically.

In a further aspect, the gas-permeable membrane materials discharge small amounts of anti-thrombogenic agent from their structure. Materials that discharge anti-thrombogenic agents include materials that release heparin and materials that release nitric oxide (NO). Generally, these materials require a surface coating that is too thick for use for the membranes in the mass exchange apparatus. However, they might be useful for other parts of the respiratory aid apparatus. Recent developments include thin surface-active coatings that generate nitric oxide from the biological materials in contact with the surface. For example, they can produce a small flux of nitric oxide when in contact with blood.

Also envisaged are gas-permeable membrane materials that combine two or more of the above properties.

Some surface treatments bind preferentially to specific substrates. Thus, in order to obtain the desired anti-coagulant surface, the choice of (substrate) membrane materials may be limited. Conversely, in order to obtain the desired diffusive properties, the choice of base materials may be limited. It is desirable to achieve an optimal compromise between diffusive and anti-coagulant properties for the membrane materials.

Together with high diffusivity and good blood compatibility, the membrane materials desirably exhibit adequate physical strength. Highly diffusive materials tend to be soft. Thus, in one aspect there is employed a thin layer of diffusive material backed by a strong mesh or microporous material. The strong mesh might be provided by an aramid fibre (for example, the product Kevlar, manufactured and sold by Dupont Inc) or by Carbon fibre.

Particular gas-permeable membrane materials for the walls include those described in European Patent Application No. 1,297,855 in the name of Dainippon Ink & Chemicals. Thus, the materials suitably comprise a hollow fibre membrane comprising poly-4-methylpentene-1 and having an oxygen permeation rate Q(O₂) at 25° C. of from 1×10⁻⁶ to 3×10⁻³ (cm³(STP)/cm².sec.cmHg) and an ethanol flux of from 0.1 to 100 ml/min.m², wherein said membrane has (e.g. in the side of the blood flow) a surface comprising an ionic complex derived from:

quaternary aliphatic alkylammonium salts; and
heparin or a heparin derivative, and
wherein said quaternary alkylammonium salts comprise a quaternary aliphatic alkylammonium salt having from 22 to 26 carbon atoms in total and a quaternary aliphatic alkylammonium salt having from 37 to 40 carbon atoms in total.

Suitably, the quaternary alkylammonium salt comprises from 5 to 35% by weight of a quaternary aliphatic alkylammonium salt having from 22 to 26 carbon atoms in total and from 65 to 95% by weight of a quaternary aliphatic alkylammonium salt having from 37 to 40 carbon atoms in total.

Suitably, the quaternary aliphatic alkylammonium salt comprises a dimethyldidodecylammonium salt or a dimethyldioctadecylammonium salt.

Suitably, air and blood flows are arranged such as to provide blood oxygen/carbon dioxide relationships similar to those for natural respiration. The air sac and air vessel of the prosthetic lung herein assist in achieving this relationship because they enable the gas carbon-dioxide concentration to be controlled.

In one aspect, the air flow pattern is a combination of counter-current to the blood flow and co-current to the blood flow and may include recycled air flow. A recycle can be achieved by discharging to atmosphere only part of the gas in the air vessel cavity. The next breath then creates a recycle by drawing in air that was passed through the mass exchange apparatus on the previous breath.

In another aspect, the air flow is mainly counter-current (i.e. in the opposite flow sense) to the blood flow.

The blood/air mass exchange apparatus herein is a counter-diffusion device that functions to transfer oxygen from the air into the blood and carbon dioxide from the blood to the air. In the air/blood mass exchange apparatus, blood and air flow in alternate channels suitably defined between a series of plates that are separated by a small distance. Suitably, the spacing between the plates is less than 0.5 millimetres, preferably from 0.2 to 0.05 millimetres.

The plates are gas-permeable membranes allowing oxygen and carbon dioxide to diffuse in opposite directions. Alternative arrangements with channels or tubes of various cross-sections are possible. The blood flows in a first direction through the apparatus. Air may flow in alternate directions (as in normal breathing); counter-current to the airflow; intermittently counter-current; co-current or intermittently co-current to the airflow. The total mass-exchange area is a fraction of the area found in a living human lung. Thus, it is expected to be of the order of from 5 to 25 square metres, for example about 20 square metres compared to 70 square metres that is typically found in a human lung. Where more than one mass exchange apparatus herein, are used together the total mass exchange area is divided between the apparatus. For example, where two apparatus are used in tandem (one for each lung), the total mass exchange area provided by these two in combination should be from 5 to 25 square metres.

A total mass-exchange area of from 5 to 25 square metres is a multiple of the area conventionally found in blood oxygenators used as part of heart/lung devices for thoracic surgery. Such blood oxygenators typically provide less than one square metre of surface area. The apparatus herein typically employs a larger area because it employs air (giving a lower mass transfer driving force) instead of oxygen, and is intended for long term use (months to years) by a conscious, mobile patient. The prosthetic lung herein is intended as an alternative to a lung transplant. Hence, it must use natural air rather than 100% oxygen as typically employed in thoracic surgery oxygenators or Extracorporeal Life Support (ECLS) devices. Use of natural air provides the three components (inert gas, nitrogen, oxygen and carbon dioxide) necessary for control of mass transfer rate, and confers light weight and mobility rather than requiring the use of enhanced oxygen concentrations that require an oxygen supply (e.g. provided as a weighty oxygen cylinder).

The prosthetic lung herein is provided with an air sac defining an air sac cavity and an air vessel defining an air vessel cavity. The air sac and air vessel may in aspects, be separate entities or share certain common walls or other common structural features or form part of an integral structure.

The principal function of the air sac is to provide a means for allowing air flow to be achieved through the mass exchange apparatus of the prosthetic lung by patient manipulation thereof (e.g. in a bellows-like action). The air sac therefore suitably comprises wholly or partly of elastic material. The principal function of the air vessel is to define a ‘dead space’. The air vessel therefore suitably comprises wholly or partly of rigid material.

In more detail, the air sac defines an air sac cavity in fluid communication with the at least one first air port of the mass exchange apparatus.

The air vessel defines an air vessel cavity in fluid communication with the at least one second air port of the mass exchange apparatus. The air vessel is also provided with an air access port that is arranged in use, to enable fluid communication with the trachea of the patient. Thus in use, air flow may be established between the trachea (and hence nose and mouth) of the patient and the air vessel cavity (and hence, the mass exchange apparatus) via the air access port.

The air sac cavity is in fluid communication with the air vessel cavity via the (at least one first and second air port of) the mass exchange apparatus. In preferred embodiments, the air vessel cavity may only fluidly communicate with the air sac cavity via the mass exchange apparatus (e.g. directly or via tubing).

The arrangement of the air sac and air vessel is arranged to supply (e.g. to draw or drive) air flow to the air flow conduits of the mass exchange apparatus such that oxygen/carbon dioxide exchange may occur with the blood flow of the blood flow conduits of the mass exchange apparatus. In aspects, the air sac functions as bellows means that act such as to supply (e.g. draw or drive) air flow through the air flow conduits. In use, the air sac is suitably arranged for manipulation by the patient through their natural breathing reflex (e.g. by manipulation of the patient's diaphragm) such as to achieve the necessary air flow through the mass exchange apparatus.

In embodiments, the air sac is arranged for receipt of the mass exchange apparatus such that the mass exchange apparatus locates within the air sac. In other embodiments, the air sac and air vessel are arranged for receipt of the mass exchange apparatus such that part of the mass exchange apparatus locates within the air sac and part within the air vessel or alternatively, locates wholly within the air sac, which suitably also encloses the air vessel.

In preferred embodiments, the air sac is comprised wholly or partly of an elastic (or flexible) material, which typically comprises a plastic polymer or rubber material. Suitable elastic air sac materials include silicone rubbers.

In preferred embodiments, the air vessel is comprised of a material that is less elastic (e.g. somewhat or wholly rigid) than the material of construction of the air sac. Suitable air vessel materials include harder silicone rubbers or other harder synthetic or natural polymers.

In embodiments, the air vessel defines an air vessel cavity of essentially fixed volume.

In embodiments, the air vessel and air sac are defined by an integral structure that is provided with a dividing wall, which divides off the air vessel from the air sac. The dividing wall may be curved in three dimensions. The dividing wall is suitably comprised of an inelastic material, and which in aspects corresponds to the material of construction of the wall(s) of the air vessel itself. However, where it joins to a flexible air-sac wall, there must be a flexible connection to accommodate the movement of the air sac during breathing.

The dividing wall acts such as to partly define an air vessel cavity and an air sac cavity within the integral structure. The air vessel cavity is arranged for fluid communication with the at least one first air port and the air sac cavity is arranged for fluid communication with the at least one second air port.

In other embodiments, the air sac wholly or partly encloses the air vessel, which effectively defines an inner compartment thereof. The air sac cavity is thus, essentially defined by the space between the inner compartment and the air sac. In use, the air vessel defining the inner compartment does not contact either blood or the chest cavity. Thus, biocompatibility is not a major consideration and there is a wide choice of possible materials of construction of the air vessel.

In embodiments, the air vessel defines an open volume, which in use suitably sits within the upper part of the pleural cavity of a patient such as to allow air flow communication with the trachea of the patient. Part of the air vessel defining the air vessel cavity may connect with the trachea of the patient. One objective of this air vessel cavity is to retain some of the spent air discharged into it from the mass exchange apparatus. Resulting from this retention, the next “in” breath through the mass exchange apparatus contains a significant concentration of carbon dioxide. By sizing the volume suitably, the concentration of carbon dioxide can be controlled such that the blood gas concentration of carbon dioxide mimics the concentration obtained with natural lungs. At the same time, the concentration of oxygen is depressed and the mass exchange apparatus is sized such that, at rest, a desired oxygen mass transfer rate is achieved. With this design, blood gas concentrations respond naturally to faster and deeper breathing. Such breathing exchanges more of the air in the air vessel cavity with the outside air. Consequently, the proportion of spent air is reduced and the concentration of carbon dioxide decreased as the concentration of oxygen is increased. On each “in” breath, there are then larger driving forces in the mass exchange apparatus and hence enhanced mass transfer rates for both oxygen and carbon dioxide. In this way, automatic control of mass transfer rates and blood gas concentrations can be achieved without the use of electromechanical devices. More subtle control of the response to increased respiratory demand can be achieved by design of the shape of the air sac and air vessel, by suitable internal baffling, and by use of fluidic components to control the flow patterns.

In use, the air sac exactly fills the space that is normally taken by the lung. It thus responds to the normal breathing reflex in exactly the same way as a natural lung. On the “in breath”, the air sac is manipulated by the patient (e.g. by diaphragm movement) such that the effective volume of the air sac cavity expands such as to draw air through the air conduits of the mass exchange apparatus. In more detail, the volume of the air sac cavity expands such as to draw air through at least one first air port, and hence also through the air conduits of the mass exchange apparatus and the at least one second air port from the air vessel. Conversely, on the “out breath”, the effective volume of the air sac cavity contracts such as to drive air from the air sac cavity through the air conduits of the mass exchange apparatus into the air vessel cavity. The air discharged to the air vessel cavity is partially spent air because it has already been drawn through the mass exchange apparatus on the “in” breath. On the “out” breath, the air is further spent in its passage back from the air sac cavity, through the mass exchange apparatus, to the air vessel cavity. The air vessel fluidly communicates with the trachea of the patient, and hence via the nose and mouth of the patient to the atmosphere.

Considering use aspects in more detail, it is helpful to define the sum of the volume of the air vessel and the inclusive volume from the trachea to the atmosphere as volume V₁. The tidal volume in the lungs of a normal healthy patient is the volume of air (at blood temperature and saturated with water vapour) that is drawn into the lung on each breath. For a healthy young male patient at rest, it is about 250 ml (that is a total of 500 ml for the two lungs together). Air is drawn in by muscle movement, primarily (under resting conditions) by contraction of the diaphragm. Air is driven out of the lungs mainly by the elastic contraction of the lungs, and lung walls, when the diaphragm relaxes. In use, each prosthetic lung herein is suitably arranged to take up exactly the same space as a natural lung of a patient. The air entering the prosthetic lung herein comes from the nose or mouth of the patient, as for natural lungs. Consequently, it is at blood temperature and saturated with water vapour. In the prosthetic lung herein, the effective volume of the air vessel cavity (and hence, of V₁) is suitably fixed and the effective volume of the air sac cavity is suitably elastic. The only volume capable of change in the natural lungs is the volume of air. Hence, the same amount of muscle movement will produce the same volume change in the natural and the prosthetic lung; an identical amount of air will be drawn in or expelled. Herein, the effective volume of the air vessel cavity is suitably greater than the tidal volume, and the elasticity of the prosthetic lung is similar to the natural lung. With this design, the air inhalation will be the same as the air inhalation for a natural lung.

In greater detail, volume V₁ is selected such that, in normal inhalation, only a proportion is exchanged with the outside atmosphere. Thus, if V₁ is initially full of air, breathing causes the concentration of carbon dioxide to rise and the concentration of oxygen to fall. For a given respiratory demand, the concentrations will ultimately cycle around an equilibrium level that depends on the breathing rate, the blood circulation rate, and the relative sizes of the tidal volume and volume V₁. Note that these equilibrium concentrations are independent of the effective volume of the air sac cavity. The design constraint on the effective volume of the air sac cavity is that it should be sufficiently large to accommodate the deepest breathing that will arise.

Thus, in response to increased respiration rates, deeper or faster breathing causes a greater proportion of the gas in V₁ to be replaced by atmospheric air. Thus, the concentration of oxygen increases and the concentration of carbon dioxide decreases. The result is a higher driving force and increased mass transfer rates. Thus, the prosthetic lung herein responds qualitatively in the same way as a natural lung. The natural respiratory control mechanism is self-tuning. Thus, it adjusts itself to compensate for lung damage, lung repair, or lung transplant. It is anticipated that these natural control mechanisms will tune themselves to compensate for relatively small quantitative differences between the prosthetic lung performance and the natural lung performance. In this way, the balance of the volumes of the air vessel cavity and air sac cavity can be selected (or tuned) to give a prosthetic lung that substitutes effectively for a natural lung. In particular, it provides higher mass transfer rates, and lower carbon dioxide concentrations, in response to increased respiratory demand. The design constraint on the volume of the air vessel cavity is that it should give desired mass transfer rates and blood gas concentrations at rest. The mass exchange area and volume must balance to give a response to higher respiratory demand that mimics the response of natural lungs.

In aspects, the prosthetic lung is arranged such as to provide access to the air sac cavity for cleaning thereof. The prosthetic lung herein has no ciliary action, and hence it is advantageous to provide means to remove any accumulated debris in the air sac cavity. Suitably, access should be using a device that does not require a surgical operation. In aspects, a cleaning device (e.g. a fine tube) is passed down the trachea, through the bronchus of the patient, and through a self-sealing opening between the air vessel cavity and air sac cavity (e.g. through a self-sealing opening provided to a dividing wall therebetween) within the prosthetic lung. In aspects, such a cleaning tube could also clean the air vessel cavity. As an alternative to a self-sealing opening, a small opening could be provided to the air sac. The flow area through each mass exchange apparatus is of the order tens of square centimetres. An opening of a few square millimetres would take such a small flow that no seal would be required.

Suitably, in normal use (when the patient is sitting or standing) the air flow through the mass exchange apparatus is essentially vertical. Vertical flow minimizes the accumulation of debris within the mass exchange apparatus. Any accumulation of debris could result in poorer distribution of air flow through the mass exchange apparatus and hence reduce its effectiveness. The effect would be similar to the degradation of performance known as “shunt” in natural lungs.

The dynamic range of the prosthetic lungs may be enhanced by providing one or more fluidic valves (or other switching means) between the air vessel cavity and the air sac cavity (e.g. at the dividing wall). The fluidic valves are suitably arranged to give more subtle control of oxygen and carbon dioxide concentrations.

The one or more fluidic valves may be suitably be arranged to allow for partial bypassing of the mass exchange apparatus by the induced air flow at either high or low breathing rates. Additionally, the one or more fluidic valves may connect by internal tubing to a supply of air taken from nearer (or within) the trachea (the left or right bronchus), so that a higher proportion of atmospheric air is drawn in at high breathing rates. This modification suitably provides for high oxygen concentrations under high breathing rates. The fluidic valves may be arranged to respond to gas velocity. Higher velocities arise both for faster and for deeper breathing.

The prosthetic lung described herein has a distinct purpose compared to a heart/lung machine in that it is intended to be permanently connected within a patient who is conscious and mobile.

The small size of the mass exchange apparatus herein is possible because fresh air is contacted directly with the membranes. This arrangement increases the driving force (and hence rate) of mass transfer by a factor approaching five compared to the human lung in which the air sacs thereof are at the end of long narrow passageways within the lung.

The mass-exchange apparatus of the present invention is suitably designed for long-term, maintenance-free operation. The straight passages, with relatively high air velocity are suitably designed to be largely self-cleaning. This self-cleaning characteristic is important because prosthetic lungs will not have the ciliary action found in living lungs.

The mass-exchange apparatus of the present invention suitably employs indirect gas/liquid contact.

Applicant has appreciated that counter-current air flow maximizes mass transfer rates in a mass exchange apparatus of a given area. However, counter-current flow disproportionately increases the efficiency of carbon dioxide mass transfer. Accordingly, co-current flow and/or recycle and/or alternating flow directions may be included to match the natural carbon dioxide/oxygen relationship in the blood. In this way, the body's natural respiratory control mechanisms operate normally. Normal operation of the control mechanisms (primarily sensing carbon dioxide levels) has the benefit that the natural control mechanisms for the metabolic system as a whole operate normally and correctly.

Fluidics is a possible method of achieving the desired flow patterns throughout the breathing cycle. A number of known fluidic devices have no moving parts so that very low maintenance would be required even for this more complex flow arrangement.

In the prosthetic lung herein, the mass exchange apparatus is connected directly to the blood circulation, so that the heart pumps blood through it in the same way that it does natural lungs. The natural lungs are removed and each lung replaced with a prosthetic lung herein. Each air sac is placed in the pleural cavity from which a natural lung has been removed. The natural breathing action expands and contracts the air sac so that it draws air through the mass exchange apparatus. No blood circulates through the air sac or air vessel, which can be designed to be rugged and maintenance-free.

The air sac of the prosthetic lung herein typically has a volume of 5 litres and delivers between 0.5 and 2 litres of air on each breath. Thus, there remains sufficient space within the air sac to install a mass exchange apparatus for each “lung”. In order to accommodate a mass exchange apparatus in each lung-space, the total volume of each mass exchange apparatus must be less than about 3 litres. From a weight viewpoint, the aim will be to provide sufficient mass transfer surface in a significantly smaller volume. The air vessel either will connect directly to the trachea (when there will be an engineered division between the two lungs) or will connect to the bronchi after they have divided from the trachea.

Benefits provided by a prosthetic lung of this form include:

1. There are no moving parts (other than elastic expansion and contraction of the air sacs). The heart provides the blood circulation. The patient's own breathing action provides the required manipulation of the air sac and hence, air flow.
2. Control can be achieved without moving parts or any electromechanical equipment. The patient's natural reflexes will cause the heart and breathing rate to match their oxygen requirements. The natural control action senses carbon-dioxide levels in blood. If it is high, respiration increases; if it is low, respiration decreases. It follows that ultra-precise design is not required. The body will automatically adjust how hard it works to the efficiency of the prosthetic lungs. (The same behaviour occurs in nature if living lungs are damaged). If efficiency deteriorates over the years, the body just works harder to accommodate the changes.
3. Pre-warmed humidified air is provided by the body's natural systems.
4. The design has no moving parts or electromechanical equipment and hence provides a long maintenance free life. This low-maintenance characteristic is important in prosthetic lungs because all significant maintenance would require a clinical procedure.

The form of the prosthetic lung herein has similarities with the lungs of birds. Birds breathe by, in effect, operating a bellows that draws air through a rigid matrix in which the counter-diffusion takes place. In the context of the prosthetic lung, this arrangement has the advantage that the matrix can be constructed from a simple arrangement of straight conduits (e.g. in plate form). For example, the matrix could be constructed from several hundred (up to a few thousand) thin parallel sheets. Blood and air would flow through alternate sheets, similar to a plate and frame heat exchanger. A similar effect could be achieved with an arrangement of fine tubes (either circular, or non-circular in cross-section). Either the blood or the air could flow through the tubes, depending on the detailed design. This construction (either sheets or tubes) solves several problems. First, sizes are within achievable robust engineering construction limits (materials can be up to around 0.1 mm thickness). Secondly, straight flow channels can allow self-clearing without ciliary action. Thirdly, the relatively high air velocity and oxygen concentration through the channels gives enhanced mass exchange requiring a smaller surface area for the same lung performance. These prosthetic lungs would have no moving parts, and no control mechanism would be required. The body's natural control action would apply. Thus, the brain senses blood carbon dioxide concentration and causes the heart and breathing rate to respond appropriately. There is the further benefit that the conduits could be mass-produced and assembled to meet the size requirements of individual patients.

The major performance differences between the proposed prosthetic lung and known heart-lung machines and ECLS devices are that the prosthetic lung has small size for ready portability; a maintenance-free design life of years rather than hours; and no intrinsic requirement for “heart” action.

The prosthetic lung herein is suitable for use with a human or animal (particularly mammalian) subject. Installation and/or use are typically under the control of a physician or veterinary surgeon. Use of the lung is however, suitably under the control of the patient without the need for any electronic controls or external connections.

### BRIEF DESCRIPTION OF THE DRAWINGS

The present invention will now be described further with reference to the accompanying drawings, in which: —

FIG. 1 shows a schematic representation of an air/blood mass exchange apparatus suitable for use with the prosthetic lung herein;

FIG. 2 shows a schematic sectional representation of a first prosthetic lung herein within the body of a patient;

FIG. 3 shows a schematic sectional representation of a second prosthetic lung herein within the body of a patient;

FIGS. 4a to 4c show schematic representations of fluidic components suitable for use herein;

FIG. 5 shows a schematic sectional representation of a prosthetic lung herein, which incorporates fluidic components;

FIG. 6 shows a schematic sectional representation of a prosthetic lung herein, which incorporates fluidic components; and

FIG. 7 shows a schematic sectional representation of a prosthetic lung herein, which incorporates a cleaning system.

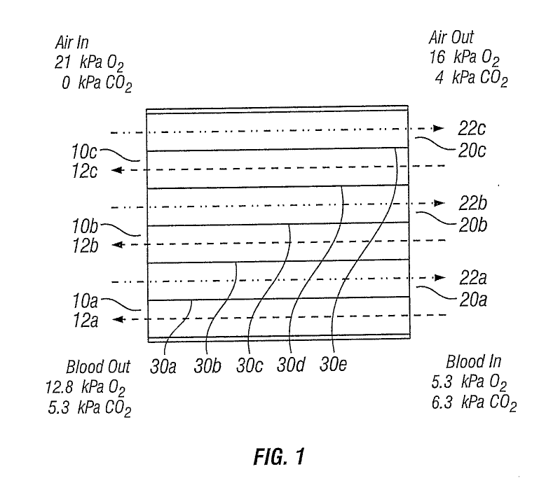

Referring now to the drawings, FIG. 1 illustrates an air/blood mass exchange apparatus herein comprising plural blood flow conduits 10a to 10c for defining blood flow 12a to 12c; and plural air flow conduits 20a to 20c for defining air flow 22a to 22c. It may be seen that the blood 12a-c and air flow 22a-c is in alternate channels defined by a series of plates 30a-e separated by less than 0.5 millimetres. Whilst for the purposes of representation, FIG. 1 shows a relatively small number of channels it will be appreciated that the actual apparatus will comprise several thousand channels to give an overall mass transfer area of from 5 to 15 square metres.

The blood flows in a first direction 12a-c through the apparatus. As shown, the air flows in a second direction 22a-c counter to the first direction. In aspects, air may flow in alternate directions (as in normal breathing), co-current to the air flow, intermittently co-current to the air flow, counter-current to the air flow, or intermittently counter-current to the air flow. Particularly, the air flow 22a-c may be arranged to be a combination of air flow 22a-c that is counter-current to the blood flow 12a-c and air flow 22a-c that is co-current to the blood flow 12a-c. The plates 30a-e are gas-permeable membranes that enable transfer of oxygen from the air to the blood and transfer of carbon dioxide from the blood to the air through said membrane material. FIG. 1 also recites typical partial pressures for oxygen and carbon dioxide. In aspects, the apparatus may additionally be provided with flow headers and dividers in accord with conventional heat exchanger design practice.

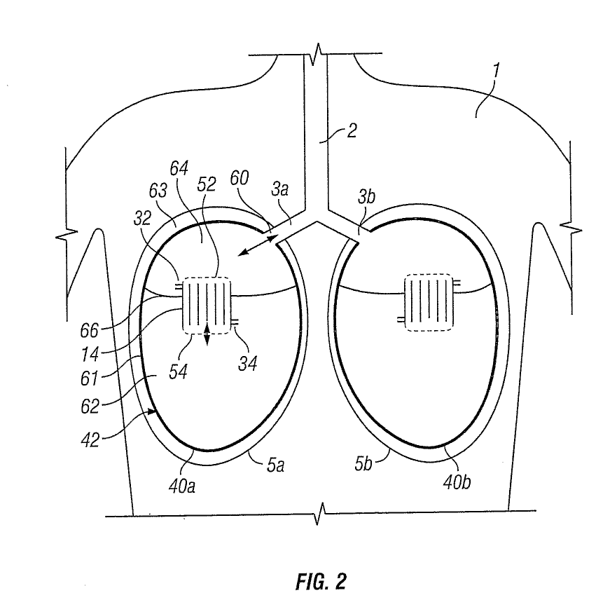

FIG. 2 illustrates in cutaway view a first patient 1 having a trachea 2 leading to the left and right bronchi 3a, 3b. Both of the patient's lungs have been removed and within the left and right pleural cavity 5a, 5b there has been ‘transplanted’ a first prosthetic lung 40a, 40b in accord with the present invention. The structure of the left-hand first prosthetic lung 40a is now described in detail (that of the right hand prosthesis is a mirror image).

The first prosthetic lung 40a comprises an integral air sac/vessel structure 42 sized and shaped for receipt by the lung cavity 5a. Within the air sac/vessel structure 42 there is provided an air/blood mass exchange apparatus 14 herein comprising plural blood flow conduits for defining blood flow and plural air flow conduits for defining air flow (detail not shown, but corresponds to that of FIG. 1). To enable an air flow to be established, within the plural air flow conduits the mass exchange apparatus 14 is provided with plural second air ports 52 and plural first air ports 54. It will be appreciated that in use, air flow may thereby be defined between the plural second air ports 52 and the plural first air ports 54 via the plural air flow conduits.

The integral air sac structure 42 is divided into an air sac 61 defining an air sac cavity 62 and an air vessel 63 defining an air vessel cavity 64 by a dividing wall 66. It will thus, be appreciated that the dividing wall 66 also forms part of the wall structure of each of the air sac 61 and the air vessel 63. The air vessel 63 is also provided with an air access port 60 arranged in use, to enable air flow communication with the trachea 2 of the patient 1.

In use, the patient 1 will control air flow to the prosthetic lung 40a by means of the same instinctive chest motion that drives living lungs. Thus, the integral structure 42 will be alternately expanded and compressed. The integral structure 42 will contract under its own elasticity (as do living lungs) and will be expanded by muscular action. During the lung expansion part of the cycle, the pressure within the integral structure 42 will fall below atmospheric pressure causing air to flow into the air vessel cavity 64 through the air access port 60 and thence, through the plural second air ports 52 of the mass exchange apparatus 14 via the plural air flow conduits and plural first air ports 54 to the air sac cavity 62. During the contraction part of the breathing cycle, the integral structure 42 is pumped causing air to flow from the air sac cavity 62 through the plural first air ports 54 of the mass exchange apparatus 14 via the plural air flow conduits and plural second air ports 52 to the air vessel cavity 64 and thence, to the trachea 3 of the patient 1 through the air access port 60. Thus, two way air flow is enabled within the mass exchange apparatus 14.

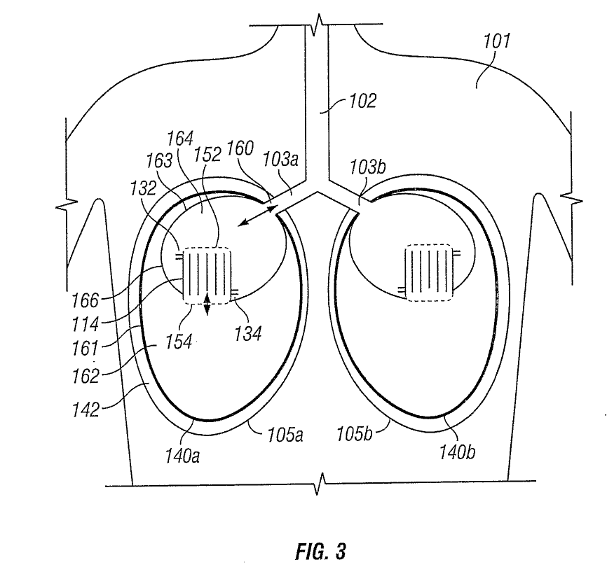

FIG. 3 illustrates in cutaway view a second patient 101 having a trachea 102 leading to the left and right bronchi 103a, 103b. Both of the patient's lungs have been removed and within the left and right pleural cavity 105a, 105b there has been ‘transplanted’ a second prosthetic lung 140a, 140b in accord with the present invention. The structure of the left-hand prosthetic lung 140a is now described in detail (that of the right hand prosthesis is a mirror image).

The second prosthetic lung 140a comprises an elastic air sac 161 sized and shaped for receipt by the lung cavity 105a. Within the elastic air sac 161 there is provided an air/blood mass exchange apparatus 114 herein comprising plural blood flow conduits for defining blood flow and plural air flow conduits for defining air flow (detail not shown, but corresponds to that of FIG. 1). To enable an air flow to be established, within the plural air flow conduits the mass exchange apparatus 114 is provided with plural second air ports 152 and plural first air ports 154. It will be appreciated that in use, air flow may thereby be defined between the plural second air ports 152 and the plural first air ports 154 via the plural air flow conduits.

The elastic air sac 161 defines an air sac cavity 162. Within and wholly enclosed by the elastic air sac 161 there is disposed an air vessel 163 defining an air vessel cavity 164. The air vessel 163 is formed of a rigid material and the air vessel cavity 164 is therefore of essentially fixed volume. The volume of the air sac cavity 162 is not fixed and will be appreciated to be essentially defined by the space between the walls of the air sac 161, the air vessel 163 and the mass exchange apparatus 114. The air vessel 163 is also provided with an air access port 160 arranged in use, to enable air flow communication with the trachea 102 of the patient 101.

In use, the patient 101 will control air flow to the prosthetic lung 140a by means of the same instinctive chest motion that drives living lungs. Thus, the elastic air sac 161 will be alternately expanded and compressed. The elastic air sac 161 will contract under its own elasticity (as do living lungs) and will be expanded by muscular action. During the lung expansion part of the cycle, the pressure within the elastic air sac 161 will fall below atmospheric pressure causing air to flow into the air vessel cavity 164 through the air access port 160 and thence, through the plural second air ports 152 of the mass exchange apparatus 114 via the plural air flow conduits and plural first air ports 154 to the air sac cavity 162. During the contraction part of the breathing cycle, the elastic air sac 161 is pumped causing air to flow from the air sac cavity 162 through the plural first air ports 154 of the mass exchange apparatus 114 via the plural air flow conduits and plural second air ports 152 to the air vessel cavity 164 and thence, to the trachea 103 of the patient 101 through the air access port 160. Thus, two way air flow is enabled within the mass exchange apparatus 114.

In the absence of fluidics, the following flow patterns are possible in the first and second prosthetic lungs of FIGS. 2 and 3 respectively. The inlet breath may be counter-current to the blood flow 12a-c, and the outlet breath co-current. This arrangement maximizes mass transfer rates. Alternatively, the inlet breath may be co-current with the blood flow 12a-c, and the outer breath counter-current. This arrangement disproportionately reduces the efficiency of carbon dioxide mass transfer. Mass transfer will take place in the mass transfer apparatus 14; 114 during both parts of the cycle, but will be more effective on the “in” breath. As a further alternative, the air flow may be controlled by fluidic switches so that air-flow patterns are achieved that give O₂/CO₂ relationships more closely mimicking the natural relationships. In this case, it might be required to divide the mass exchange apparatus into parts with distinct flow patterns in each part.

The patient's blood flows into the mass exchange apparatus 14; 114 by means of blood inlet 32; 132 and exits via blood outlet 34; 134. It will be appreciated that the blood flow inlet 32; 132 and outlet 34; 134 will be connected to the patient's blood supply and that flow will be governed by the pumping action of the patient's heart (not shown). The flow headers to divide the fluid flows between the channels and to keep the two fluids separate will be similar to those in a conventional heat exchanger, and are not illustrated.

### Fluidic Components

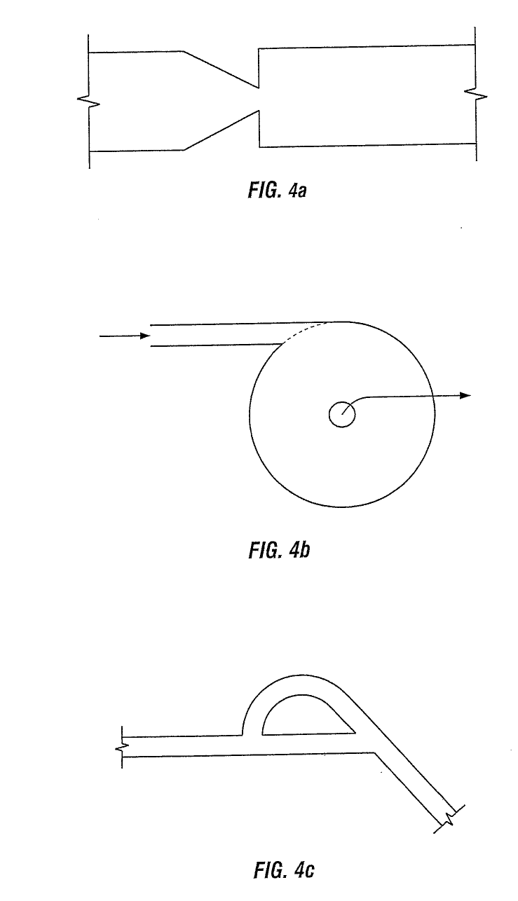

The prosthetic lungs herein may optionally incorporate fluidic components. Three suitable fluidic rectifiers are illustrated in FIGS. 4a to 4c. These have non-linear flow characteristics. Thus, at low flow rates they have negligible resistance to flow in both directions. At higher flow rates, the flow resistance in one direction becomes much higher than in the other direction. Thus, they are not strictly “rectifiers”, rather at sufficiently high flow rate they place a high resistance to flow in one direction. The flow rate at which the resistance becomes significant depends on the size and detailed design of the fluidic device.

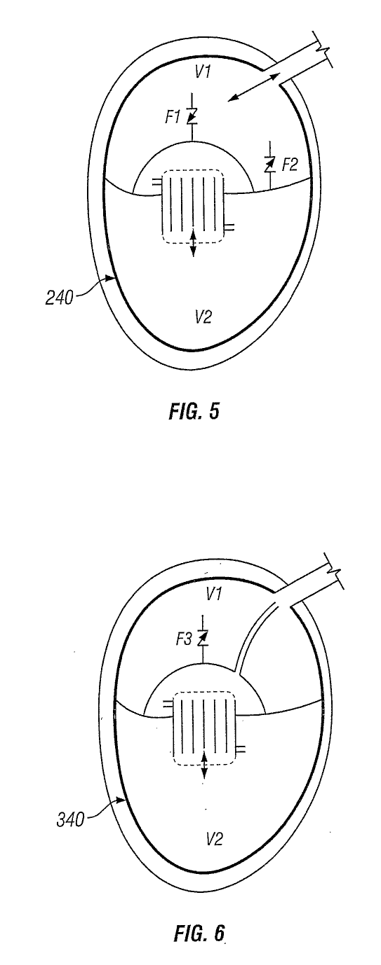

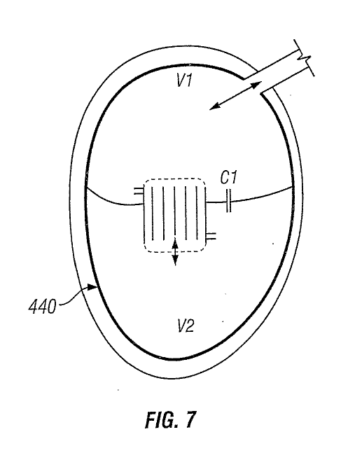

In the prosthetic lungs herein, these fluidic rectifiers can be employed either to direct the flow so that it is predominately in one direction, or to direct flow through alternative channels, depending on the flow rate. FIGS. 5 and 6 illustrate these two applications.

FIG. 5 shows two fluidic rectifiers, F1 and F2 located within a prosthetic lung 240 herein. On the “in” breath, there is a small resistance through one and a larger resistance through the other. Conversely, on the out breath flow through the other device is favoured. The outcome is that, in one direction, the flow is predominately through the mass exchange apparatus. In the other direction, the flow predominately bypasses the mass exchange apparatus. In this way, the flow through the mass exchange apparatus becomes intermittent, but almost unidirectional.

FIG. 6 shows one valve-like fluidic rectifier, F3 located within a prosthetic lung 340 herein. In FIG. 6, fluidic rectifier F3 shows high resistance to flow from volume V1 to volume V2 at high flow rates. At low flow rates, the resistance in both directions is very low. Thus, at low flow rates (e.g. resting breathing), the flow is in alternate directions through the valve F3, and there is limited flow through the tube leading directly to the trachea. This limited flow is achieved by suitably sizing the tube, or by incorporating a flow resistance. However, at high respiration rates, the flow resistance through valve F3 becomes significant on the “in” breath. Relatively fresh air is then drawn through the tube communicating with the trachea. This air is not diluted with the spent air discharged to volume V1, and hence has a higher oxygen concentration and a lower carbon dioxide concentration. In this way, there are larger driving forces and higher mass transfer rates at high respiratory demands.

### Cleaning Systems

FIG. 7 shows a prosthetic lung 440 herein provided with a cleaning opening C1. This is a very small opening in the inner vessel. If it has an area of at most a few square millimetres, it will take less than 0.1% of the flow through the mass exchanger. It can be augmented by a guide directing a fine tube to it. In this way, a fine tube directed through the trachea can be guided into the elastic air sac (volume V2). The tube can then be used to suck out any debris, or to feed antibacterial agents to ensure that potential microbial colonies do not establish themselves in the prosthetic lung. The same tube can be used to probe the inelastic air vessel (volume V1) to ensure that it also remains clean.

A larger opening could be filled with a self-sealing material, such a soft silicone rubber.

Applicant's earlier published PCT Patent Application No. W02005/118025, which is incorporated herein by reference, describes various factors relating to (a) The function of the human lung; (b) The structure of the human lung; and (c) Mass Transfer in respiratory aids and prosthetic lungs.

In designing a prosthetic lung, it is desirable that the solution does not restrict the normal movement of the patient. The apparatus desirably requires no maintenance for tens of years and fits into the lung cavity. The apparatus should also desirably have no motor or engineered control system, and be powered only by the normal movements of the chest and diaphragm.

The air sacs suitable for use in the prosthetic lung herein are in general, two elastic sacs, one for each lung. They fill the lung cavities, each being about five litres in volume. (This volume varies considerably from person to person). The air sacs may be individually made, or could be manufactured in a range of standard sizes. The air sacs contain no blood flow and need not be thin and fragile. They can thus be extremely robust with hope for a long maintenance-free life.

The mass exchange apparatus can be made of thin sheets of gas-permeable material. The sheets may contain a high density of parallel capillary channels through which blood flows. Alternatively, they could be two sheets closely joined with a small space between to allow blood flow. In either case, the sheets carrying the blood flow would be stacked with a small air space between each. As a further alternative, the mass exchange apparatus could be made of fine tubing (“hollow fibres”) with the air flowing through or around the tubes. The air sacs would pump the air through the spaces to create effective mass-transfer conditions. As an order of magnitude estimate, a mass exchange apparatus having a volume of 3 litres would have an air space of a litre and leave the air sacs space to shift up to 2 litres of air at each breath.

The only part of the prosthetic lung that regularly moves (expands and contracts) is the air sac. This part can be made extremely robust.

The walls defining the conduits of the mass exchange apparatus are typically only a fraction of a millimetre thick. However, they will not move significantly. Thus, the exchanger will not be subject to the stresses of the alveolar air sacs, so that risk of damage is reduced. Materials of construction may be determined by gas permeability or biocompatibility considerations. Both rigid and flexible materials may be considered.

The straight air channels in the mass exchange apparatus are swept by air, therefore, we may expect them to be self-cleaning.

One important design consideration is low pressure drop. The pressure drop on the blood side should be sufficiently low that the blood can be pumped through it using normal blood pressure. The design blood-side pressure drop is suitably no more than of order 1 kPa (5 inches of water, or 10 mm Hg). The design air-side pressure drop is suitably no more than 0.1 kPa (1 inch of water, 2 mm Hg). Spacing (or tube diameters) of a fraction of a millimetre (for example, 0.1 mm to 0.2 mm) allow such low pressure-drops to be achieved. The pressure drops can be achieved whilst still meeting the target total mass exchange area within a volume of order 1 litre.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1-18. (canceled)

19. A prosthetic lung for receipt by a lung space of a patient, comprising:
a mass exchange apparatus for use in blood/air mass exchange including:
plural blood flow conduits for defining blood flow; and,
plural air flow conduits for defining air flow,
wherein said plural air flow conduits and said plural blood flow conduits at least partially comprise gas-permeable membrane material, and the conduits being relative to each other for enabling a transfer of oxygen from air to blood and transfer of carbon dioxide from the blood to the air through said gas-permeable membrane material, said mass exchange apparatus including at least one first air port and at least one second air port so that said air flow is defined between said at least one first air port to the at least one second air port via the plural air flow conduits;
an air sac defining an air sac cavity in fluid communication with the at least one first air port of said mass exchange apparatus; and,
an air vessel defining an air vessel cavity in fluid communication with the at least one second air port of the mass exchange apparatus, said air vessel having an air access port arranged for enabling air flow communication with the trachea of the patient.

20. The prosthetic lung according to claim 19, wherein said mass exchange apparatus is located within the air sac.

21. The prosthetic lung according claim 19, wherein the air sac shares at least one common structural feature with the air vessel.

22. The prosthetic lung according to claim 21, wherein the air vessel and air sac are defined by an integral air sac structure having a dividing wall for dividing off the air vessel from the air sac.

23. The prosthetic lung according to claim 21, wherein the air sac wholly or partly encloses the air vessel for defining an inner compartment thereof.

24. The prosthetic lung according to claim 19, wherein the air sac comprises an elastic material.

25. The prosthetic lung according to claim 19, wherein the air vessel comprises a rigid material.

26. The prosthetic lung according to claim 25, wherein the air sac comprises a plastic polymer material.

27. The prosthetic lung according to claim 26, wherein the air sac comprises a silicone rubber material.

28. The prosthetic lung according to claim 19, wherein the air vessel, in use, fits within the upper part of a pleural cavity of the patient for allowing air flow communication with the trachea of the patient.

29. The prosthetic lung according to claim 19, wherein the air sac is provides access to the air sac cavity for cleaning thereof.

30. The prosthetic lung according to claim 29, wherein a self-sealing opening to the air sac allows a cleaning device to pass into the air sac cavity.

31. The prosthetic lung according to claim 19, wherein the air flow through the mass exchange apparatus is substantially vertical when the patient is sitting or standing.

32. The prosthetic lung according to claim 19, wherein at least one fluidic valves are provided between the air vessel cavity and the air sac cavity.

33. The prosthetic lung according to claim 32, wherein said at least one fluidic valves is able to be connected via internal tubing to a supply of air taken from near to, or within, the trachea of a patient.

34. The prosthetic lung according to claim 19, wherein the air flow includes a combination of air flow that is counter-current to the blood flow and air flow that is co-current to the blood flow.

35. The prosthetic lung according to claim 19, wherein the blood flow conduits have a diameter of less than 0.5 millimeters.

36. The prosthetic lung according to claim 19, wherein the air flow conduits have a diameter of less than 0.5 millimeters.

37. The prosthetic lung according to claim 19, wherein the blood flow conduits and air flow conduits are defined by a series of plates that are separated by a distance of less than 0.5 millimeters.
